(19) United States
(12) Patent Application Publication
(10) Pub. No.: US 2017/0354524 A1
(43) Pub. Date: Dec. 14, 2017

(54) MULTILAYER BALLOONS

(71) Applicant: Medtronic Vascular, Inc., Santa Rosa, CA (US)

(72) Inventors: Aram Jamous, Ballybrit (IE); Colin Meade, Ballybrit (IE)

(21) Appl. No.: 15/615,128

(22) Filed: Jun. 6, 2017

Related U.S. Application Data
(60) Provisional application No. 62/347,118, filed on Jun. 8, 2016.

(51) Int. Cl. A61F 2/958; A61M 25/10; A61F 2/95

(52) U.S. Cl. CPC A61F 2/958; A61M 25/1029; A61M 25/104; A61M 25/1036; A61M 2025/1004; A61F 2002/9534; A61F 2002/9583; A61M 2025/1075

(57) ABSTRACT

In some examples, a medical device includes a balloon inflatable to an inflated configuration. The balloon includes an outer layer coextruded on an inner layer. The outer layer has a maximum radial ratio that is lower than that of the inner layer.

This application claims the benefit of U.S. Provisional Application No. 62/347,118, entitled, “MULTILAYER BALLOON OPTIMIZING MATERIAL CAPABILITIES,” which was filed on Jun. 8, 2016 and is incorporated by reference herein in its entirety.

### TECHNICAL FIELD

This disclosure relates to medical devices including balloons.

### BACKGROUND

Catheters may be used in intravascular procedures or other procedures to facilitate minimally invasive access to a target site. For example, an angioplasty catheter may include balloons mounted to the catheter that may be advanced to the target site and inflated to clear or compress a blockage, for example a stenosis. As another example, a stent delivery catheter may include a stent positioned over a balloon, which may be inflated to deploy the stent.

### SUMMARY

Example medical devices include multilayer balloons are described herein. In some examples, an example balloon includes an inner layer, and an outer layer coextruded on the inner layer. The outer layer has a maximum radial ratio that is lower than that of the inner layer.

Clause 1: In some examples, a medical device includes a balloon inflatable to an inflated configuration. The balloon includes an inner layer and an outer layer coextruded on the inner layer. The outer layer has a maximum radial ratio that is lower than that of the inner layer.

Clause 2: In some examples of the medical device of clause 1, the inner layer has a first maximum radial ratio of up to 8.5, and the outer layer has a second maximum radial ratio of up to 6.5.

Clause 3: In some examples of the medical device of clauses 1 or 2, the inner layer includes a first material having a first glass transition temperature, and the outer layer includes a second material having a second glass transition temperature higher than the first glass transition temperature.

Clause 4: In some examples of the medical device of any one of clauses 1 to 3, the inner layer has a lower Shore durometer hardness than that of the outer layer.

Clause 5: In some examples of the medical device of any one of clauses 1 to 4, the inner layer is more flexible than the outer layer.

Clause 6: In some examples of the medical device of any one of clauses 1 to 5, the balloon has a wall thickness of less than 0.0635 mm (0.0025 inches).

Clause 7: In some examples of the medical device of clause 6, the balloon has a burst pressure of at least 1013 kPa (10 atmospheres).

Clause 8: In some examples of the medical device of clause 7, the balloon has a burst pressure of at least 4053 kPa (40 atmospheres).

Clause 9: In some examples of the medical device of any one of clauses 1 to 8, the inner layer has a lower stiffness than the outer layer.

Clause 10: In some examples of the medical device of any one of clauses 1 to 9, the outer layer includes a biaxially oriented thermoplastic.

Clause 11: In some examples of the medical device of clause 10, the biaxially oriented thermoplastic includes one or more of a polyamide, a nylon 12, a nylon 6/12, a nylon 610, a nylon 612, or a nylon 1010, a polyester, a polyethelene terephthalate, or a polyurethane.

Clause 12: In some examples of the medical device of any one of clauses 1 to 11, at least one of the inner layer or the outer layer includes a thermoplastic elastomer.

Clause 13: In some examples of the medical device of clause 12, the thermoplastic elastomer includes a polyether block amide (PEBA).

Clause 14: In some examples, a system includes the medical device of any one of clauses 1 to 13 and a second medical device secured to the balloon.

Clause 15: In some examples of the system of clause 14, the second medical device includes a stent crimped to the balloon.

Clause 16: In some examples, the medical device of any one of clauses 1 to 15 further includes an elongated member. The balloon is mounted to the elongated member, and the elongated member includes a catheter body.

Clause 17: In some examples, a medical device includes a balloon inflatable to an inflated configuration. The balloon includes an inner layer and an outer layer coextruded on the inner layer. The inner layer includes a first material having a first glass transition temperature. The outer layer includes a second material having a second glass transition temperature higher than the first glass transition temperature. The balloon has a wall thickness of less than 0.0635 mm (0.0025 inches), and a burst pressure of at least 4053 kPa (40 atmospheres).

Clause 18: In some examples of the medical device of clause 17, the inner layer has a first maximum radial ratio of up to 8.5, and the outer layer has a second maximum radial ratio of up to 6.5.

Clause 19: In some examples, a method includes coextruding an outer layer on an inner layer to form an elongated tube, the outer layer has a maximum radial ratio that is lower than that of the inner layer; and forming a balloon by at least expanding the elongated tube within a mold defining a predetermined outer diameter of the balloon.

Clause 20: In some examples of the method of clause 19, forming the balloon further includes molding the inner layer and the outer layer over a scaffold.

Clause 21: In some examples of the method of clause 19 or 20, forming the balloon further includes heat-setting the balloon.

Clause 22: In some examples, the method of any one of clauses 19 to 21 further includes securing a medical device to the balloon.

Clause 23: In some examples of the method of clause 22, securing the medical device to the balloon includes crimping a stent to the balloon.

Clause 24: In some examples, a method includes introducing a balloon into vasculature of a patient, and after introducing the balloon into the vasculature, pressurizing the balloon to an operational pressure. The balloon includes an outer layer coextruded on an inner layer, the outer layer having a maximum radial ratio that is lower than that of the inner layer.

Clause 25: In some examples, the method of clause 24 further includes, after pressurizing the balloon, deflating the balloon, and withdrawing the balloon from the vasculature.

Clause 26: In some examples of the method of claim 24 or 25, the balloon may be any one of the balloons of clauses 1 to 18.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a schematic and conceptual side view of an example medical device including an elongated member and a balloon.

FIG. 2 is a schematic and conceptual partial side view of the example medical device of FIG. 1 further including a second medical device secured to the balloon.

FIG. 3 is a schematic and conceptual cross-sectional view of the balloon of FIG. 1, in an inflated configuration, where the cross-section is taken in a direction orthogonal to a longitudinal axis of the medical device.

FIG. 4 is a schematic and conceptual cross-sectional view of a mold including a tube being inflated to form the balloon of FIG. 3.

FIG. 5 is a flow diagram illustrating an example technique for preparing an example balloon.

FIG. 6 is a flow diagram illustrating an example technique for using an example balloon in a procedure.

FIG. 7 is a graph illustrating a relationship between burst pressure and wall thickness for an example single layer balloon.

FIG. 8A is a histogram illustrating wall thicknesses of a sample population of double layer balloons.

FIG. 8B is a histogram illustrating burst pressures a sample population of double layer balloons.

FIG. 8C is a histogram illustrating outer diameters of a sample population of double layer balloons.

FIG. 9 is a graph illustrating probability of burst at different pressures for balloons subjected to cycling.

### DETAILED DESCRIPTION

The disclosure describes multilayer balloons having a relatively high burst resistance at a relatively lower wall thickness compared to single layer balloons, and example techniques for forming and using multilayer balloons.

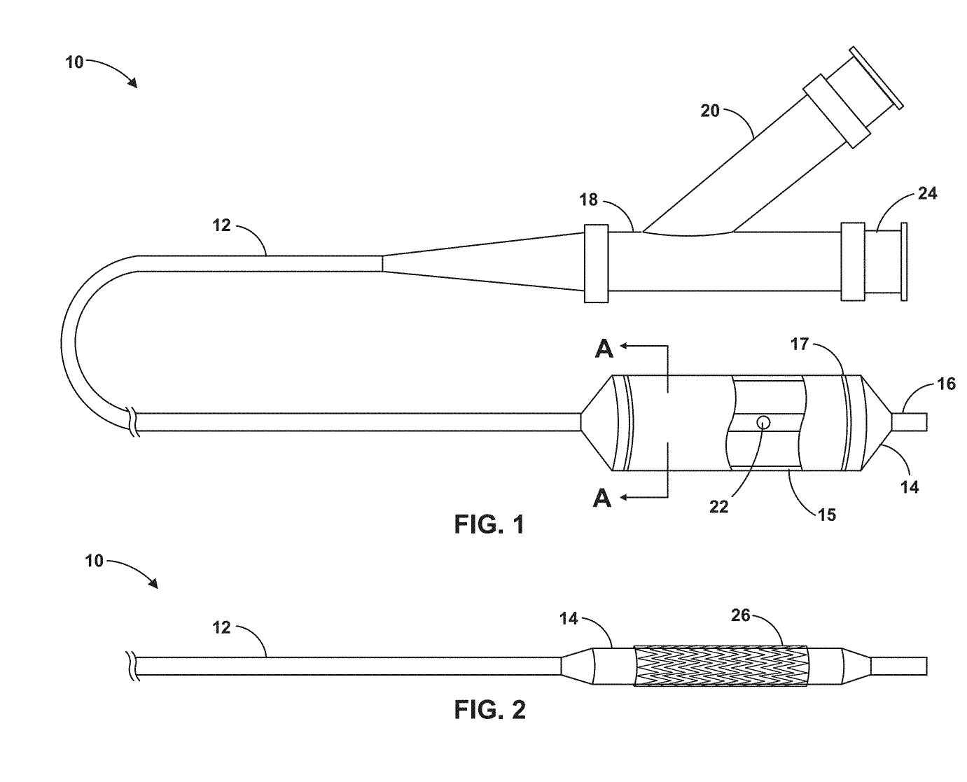

FIG. 1 is a schematic and conceptual side view of an example medical device 10 including an elongated member 12 and a balloon 14 mounted closer to a distal tip 16 of elongated member 12 than a proximal end of elongate member 12. A hub 18 connected to the proximal end of elongated member 12 may allow elongated member 12 to be manipulated, advanced, or retracted, and may provide ports for communicating with lumens defined by elongated member 12. For example, hub 18 may include an inflation arm 20 that may be connected to a source of inflating fluid to deliver inflating fluid through an inflation lumen port 22 to inflate balloon 14, or deflate balloon 14 by withdrawing the inflating fluid. In some examples, hub 18 may include an adapter 24 configured to receive a guidewire through a guidewire lumen in elongated member 12 (not shown). In some examples, elongated member 12 may include a catheter body, for example, a balloon catheter, and hub 18 may include a catheter hub. In some examples, medical device 10 may include a rapid-exchange balloon catheter system.

Elongated member 12 may be advanced to a target site, for example, through a body lumen such as a blood vessel of a patient. In some examples, distal tip 16 may be introduced into the vasculature of the patient through an incision or opening, followed by a shaft of elongated member 12. Elongated member 12 may be advanced through the body lumen, for example, over a guidewire or another guide member (e.g., a guide catheter) introduced through adapter 24 of hub 18. In some examples, balloon 14 may be maintained in an uninflated or partly inflated configuration while advancing elongated member 12 through the vasculature. When elongated member 12 is sufficiently advanced, for example, such that balloon 14 is adjacent the target site, inflating fluid may be delivered through inflation lumen port 22 to inflate balloon 14 to an inflated configuration at the target site.

Balloon 14 is illustrated in an inflated configuration in FIG. 1. In some examples, inflation of balloon 14 may result in expansion of the vasculature, or removal of blockage, for example, clots, debris, or fat at the target site. The inflating fluid may subsequently be withdrawn through inflation lumen port 22 to result in deflation of balloon 14, and deflated balloon 14 may be withdrawn through the vasculature by retracting elongated member 12.

In some examples, balloon 14 may include one or more radiopaque markers 17. For example, radiopaque marker 17 may include one or more radiopaque bands disposed about balloon 14, such as one marker 17 adjacent the proximal end of balloon 14 and another marker 17 adjacent a distal end of balloon 14, as shown in FIG. 1. Radiopaque marker 17 may allow a clinician to observe balloon 14 using suitable radioimaging techniques during a medical procedure, for example, while advancing or retracting balloon 14 with elongated member 12.

In some examples, medical device 10 may include a second medical device 26. FIG. 2 is a schematic and conceptual partial side view of medical device 10 of FIG. 1 further including second medical device 26 positioned over (e.g., co-axial with) balloon 14, and, in some examples, secured to balloon 14.

In some examples, as illustrated in FIG. 2, second medical device 26 may include a stent crimped to balloon 14. Balloon 14 is illustrated in an uninflated configuration in FIG. 2, with the stent in an unexpanded configuration ready for deployment. In examples in which second medical device 26 is secured to balloon 14, second medical device 26 may be advanced with balloon 14 to the target site, and balloon 14 may be inflated to deploy second medical device 26. For example, in examples in which second medical device 26 includes a stent, inflation of balloon 14 may expand the stent to an expanded state to scaffold a region of the vasculature adjacent the stent. After deploying second medical device 26 at a target site within the patient and subsequently partially or fully deflating balloon 14, second medical device 26 may remain in a deployed configuration at the target site, and only balloon 14 can be withdrawn from the patient. In other examples, second medical device 26 may be withdrawn with balloon 14 after balloon 14 is partly or completely deflated.

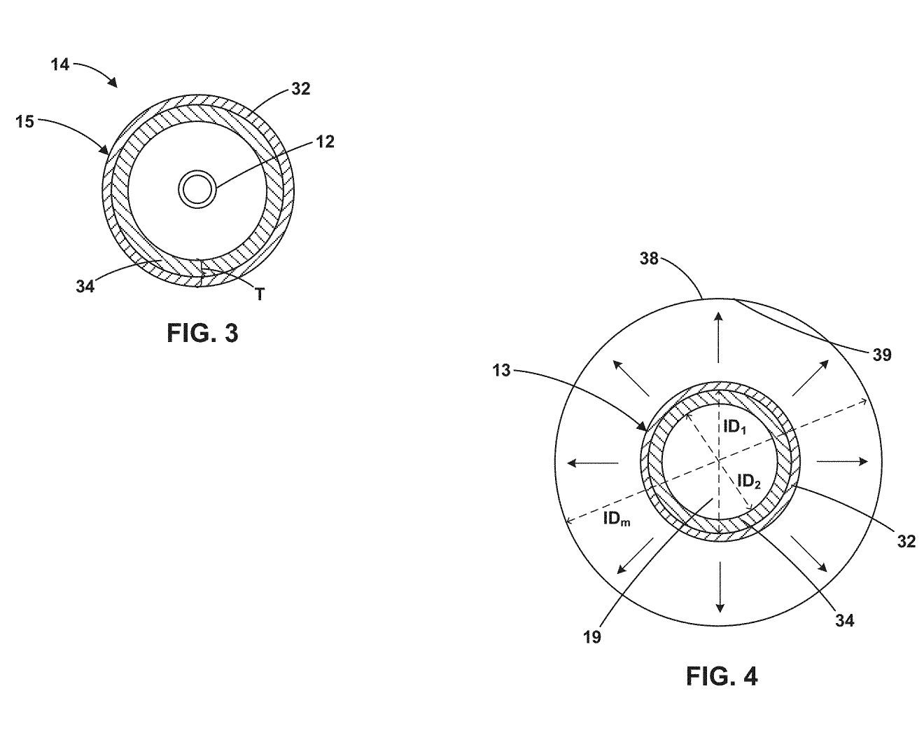

As shown in FIG. 1, balloon 14 may be defined by a balloon wall 15, which includes more than one layer, such that balloon 14 is a multilayer balloon. An example balloon 14 and balloon wall 15 are described with reference to FIG. 3, which illustrates a schematic and cross-sectional view of balloon 14, the cross-section A-A being taken in a direction orthogonal to a longitudinal axis of elongated member 12 in FIG. 1.

FIG. 3 is a schematic and conceptual cross-sectional view of balloon 14 of FIG. 1, in an inflated configuration. Balloon wall 15 includes outer layer 32 and inner layer 34. In some examples, outer layer 32 defines an outermost or exterior surface of balloon 14, while inner layer 34 defines an innermost or interior surface of balloon 14. However, in some examples, balloon 14 may include one or more additional layers, such as one or more intermediate layers between outer layer 32 and inner layer 34, one or more layers internal to inner layer 34, or one or more layers external to outer layer 32. In addition, in some examples, one or more coatings or surface treatments may be applied to outer layer 32, such as, but not limited to, a lubricious coating, a lubricious surface treatment, or a therapeutic agent (e.g., an anti-restenotic agent).

While in the examples shown in FIG. 3, balloon 14 is illustrated as having two layers, in other examples, balloon 14 may have any number of additional layers. In some examples, one or more tie layers may be provided between adjacent layers of balloon 14. A tie layer is a layer that may tie or bond adjacent layers together, for example, layers that may be incompatibly or have low joinability or adhesion. For example, one or more tie layers may be coextruded with other layers of balloon 14 to facilitate bonding of the other layers to each other. In some examples, a tie layer may include one or more materials described with reference to outer layer 32 or inner layer 34.

While in the example shown in FIG. 3, balloon 14 is illustrated having a circular cross-section, in other examples, balloon 14 may have any suitable shape, configuration, or cross-sectional shape. In some examples, balloon 14 may have a geometrically similar shape in an uninflated and an inflated configuration. For example, balloon 14 may be cylindrical in both uninflated and inflated configurations. In some examples, balloon 14 may have different shapes in uninflated and inflated configurations. For example, balloon 14 may be folded or otherwise have a compact uninflated configuration and may define a cylindrical or another shape in an inflated configuration.

Outer layer 32 and inner layer 34 may be formed of an inflatable material, for example, a polymer material that reversibly expands when subject to an inflation pressure. Outer layer 32 and inner layer 34 may have different compositions, which may provide outer layer 32 and inner layer 34 with relative differences in one or more of maximum radial ratio, compliance, stiffness, or hardness. These differences may affect properties of balloon 14, such as burst resistance, and deliverability, as described in the disclosure.

The maximum radial ratio of a layer of balloon 14 may depend on the orientability of the layer. In some examples described herein, one or more layers of balloon 14 may include one or more polymeric materials. A layer including a polymeric material may exhibit an internal orientation, for example, an orientation arising from alignment of polymer chains along substantially the same direction. For example, when a polymeric material is heated to a temperature above the glass transition temperature of the polymeric material, polymer chains in the material may substantially realign along a direction in response to stress applied along that direction. If the polymeric material is then cooled sufficiently rapidly to prevent relaxation of the polymeric chains, the alignment of the polymeric chains will be maintained. Such alignment may increase the crystallinity within a polymeric material. For example, the crystallinity of a region of a polymeric material may be indicative of the extent of orientation within that region, a relatively higher crystallinity being indicative of a relatively higher orientation. The orientation or crystallinity may be determined using any suitable technique, for example, by x-ray diffraction, birefringence analysis, or the so-called Chrysler test.

The internal orientation within outer layer 32 or inner layer 34 of balloon 14 may be affected by the heat, pressure, and stretching used to form balloon 14. In some examples, balloon 14 may be formed by expanding a tube including outer layer 32 and inner layer 34 in a mold while subjecting the tube to predetermined pressure, temperature, and radial or axial stretching. The respective internal orientations of outer layer 32 and inner layer 34 of balloon 14 may affect properties such as compliance and burst pressure of balloon 14.

The orientation of a layer of balloon 14 may be characterized in terms of a radial ratio of the layer. The radial ratio of a layer of balloon 14 is a ratio of the outer diameter of a fully expanded tube including the layer in the mold to the inner diameter of the layer (before expanding the tube in the mold during formation of balloon 14 from the tube). For example, if the outer diameter of the fully expanded tube is OD_{expanded}, and if the inner diameter of the tube before the expansion is IDᵢₙᵢₜᵢₐₗ, then the radial ratio RR can be determined as RR=OD_{expanded}/IDᵢₙᵢₜᵢₐₗ. In some examples, the outer diameter of the fully expanded tube may be substantially the same as an inner diameter defined by the mold which constrains the tube as it is expanded (which can also be referred to as “blown”). While the radial ratio of a layer of balloon 14 may depend on the conditions under which balloon 14 was formed, the radial ratio may be indicative of the structural configuration of the layer after forming balloon 14, for example, the degree of orientation of material within the layer. For example, a layer having a relatively higher radial ratio may exhibit higher orientation compared to another layer having a relatively lower radial ratio.

A maximum radial ratio of a layer is the highest radial ratio to which a layer may be expanded before it ruptures or tears during expanding. For example, if the outer diameter at which the expanded layer begins to rupture during the formation of balloon 14 from the tube is ODᵣᵤₚₜᵤᵣₑ, and if the inner diameter of the tube before the expansion is IDᵢₙᵢₜᵢₐₗ, then the maximum radial ratio RRₘₐₓ can be determined as RRₘₐₓ=ODᵣᵤₚₜᵤᵣₑ/IDᵢₙᵢₜᵢₐₗ. The maximum radial ratio RRₘₐₓ that a layer of balloon 14 is capable of enduring depends on the composition of the layer. For example, a layer having a particular composition may rupture or tear if the layer is expanded beyond a maximum radial ratio during formation of balloon 14 from the tube. A layer that has a relatively higher orientability can sustain a relatively greater expansion, because such a layer exhibits a higher degree of realignment of polymer chains, allowing for more expansion without rupture or tearing. Thus, a higher maximum radial ratio is indicative of a higher orientability, because a layer with a relatively higher maximum radial ratio can be blown to a relatively higher radial ratio without compromising the structural integrity of the layer of the balloon during expanding.

The radial ratio RR to which a layer of balloon 14 is actually subjected to without rupture or tearing during forming of the balloon may be any predetermined radial ratio RR less than the maximum radial ratio RRₘₐₓ. While the radial ratio of a layer of balloon 14 prepared by expanding a tube including the layer in a mold depends on the inner diameter defined by the mold, the maximum radial ratio of the layer is independent of the inner diameter defined by the mold. The maximum radial ratio of the layer depends on the thickness and the composition of the layer in the tube before the tube is expanded to form the balloon.

The burst resistance and ease of inflation of balloon 14 during use (e.g., during a medical procedure) may depend on the relative maximum radial ratios of outer layer 32 and inner layer 34. For example, balloon 14 may include outer layer 32 having a maximum radial ratio that is lower than the maximum radial ratio of inner layer 34. In some such examples, balloon 14 may have the same wall thickness as a single layer balloon, but have a relatively higher burst strength than the single layer balloon. A higher burst strength of a balloon can result in an ability to withstand higher inflation pressures. Alternatively, in some such examples, balloon 14 may have a wall thickness less than a walk thickness of a single layer balloon, while having the same or similar burst strength as the single-layer balloon. For example, a single layer balloon including GRILAMID L25 (a film extrusion grade of polyimide (nylon) 12, available from EMS-Grivory, Domat/Ems, Switzerland) may require a wall thickness of about 0.2667 mm (0.0105 inches) to achieve a burst pressure of about 4255.6 kPa (42 atmospheres), and balloon 14 including two or more layers (for example, outer layer 32 including GRILAMID L25, and inner layer 34 including PEBAX 7033 (a thermoplastic elastomer made of flexible polyether and rigid polyamide, available from Arkema, Paris, France) may achieve the same burst pressure with a significantly lower wall thickness such as about 0.0584 mm (0.0023 inches). For a given wall thickness, balloon 14 may have a higher burst strength than a multilayer balloon that does not include an outer layer having a maximum radial ratio that is lower than a maximum radial ratio of an inner layer.

A maximum radial ratio RRₘₐₓ of a layer of balloon 14 can be determined as RRₘₐₓ=ODᵣᵤₚₜᵤᵣₑ/IDᵢₙᵢₜᵢₐₗ, where ODᵣᵤₚₜᵤᵣₑ is the outer diameter of balloon 14 at which the expanded layer begins to rupture during the formation of balloon 14, and where IDᵢₙᵢₜᵢₐₗ is the initial inner diameter of the layer before expansion to form balloon 14. In some examples, outer layer 32 may have a maximum radial ratio lower than that of inner layer 34. For example, inner layer 34 may have a first maximum radial ratio, and outer layer 32 may have a second maximum radial ratio lower than the first maximum radial ratio. The first maximum radial ratio may be determined by the ratio RRₘₐₓ=ODᵣᵤₚₜᵤᵣₑ/IDᵢₙᵢₜᵢₐₗ for inner layer 34. The second maximum radial ratio may be determined by the ratio RRₘₐₓ=ODᵣᵤₚₜᵤᵣₑ/IDᵢₙᵢₜᵢₐₗ for outer layer 32. In some examples, the first maximum radial ratio may be greater than 2, 3, 4, 5, 6, 7, or 8, and the second maximum radial ratio may be greater than 1, 2, 3, 4, 5, or 6. For example, when the first maximum radial ratio of inner layer 34 is 6, the second radial ratio of outer layer 32 may be 5. In such an example, outer layer 32 having RRₘₐₓ=5 is capable of expanding during forming balloon 14 to provide balloon with an outer diameter of up to 5 times that of initial inner diameter IDᵢₙᵢₜᵢₐₗ of outer layer 32 (without exhibiting rupture or tearing during the formation of balloon 14). Similarly, in such an example, inner layer 34 having RRₘₐₓ=6 is capable of expanding during forming balloon 14 to provide balloon with an outer diameter of up to 6 times that of initial inner diameter IDᵢₙᵢₜᵢₐₗ of inner layer 34 (without exhibiting rupture or tearing during the formation of balloon 14). In some examples, the first maximum radial ratio of inner layer 34 may be up to 8.5, and the second maximum radial ratio of outer layer 32 may be up to 6.5. In some examples, the first maximum radial ratio of inner layer 34 may be higher than the second maximum radial ratio of outer layer 32 by at least 0.2, or 0.5, or 1, or 1.5, or 2.

Outer layer 32 and inner layer 34 may be formed from materials having different compositions to provide the different maximum radial ratios. Without being bound by theory, a material having a relatively lower glass transition temperature or lower melt temperature may be more orientable, and may have a higher maximum radial ratio, than a material having a relatively higher glass transition temperature or melt temperature. For example, inner layer 34 may include a first material having a first glass transition temperature, and outer layer 32 may include a second material having a second glass transition temperature higher than the first glass transition temperature.

In the case of polymeric materials, the glass transition temperature of a material may be indicative of the hardness or the stiffness of the material. Without being bound by theory, a layer having one or both of a lower hardness, a lower stiffness, or higher flexibility, relative to another layer may have a higher maximum radial ratio than the other layer. The hardness of a balloon layer may be the resistance of the layer to local deformation (for example, fracture, cracking, or tearing) initiated at a surface region. The stiffness of a balloon layer may be the resistance of the balloon layer to deformation by an applied force or pressure, and is indicative of the propensity of the layer to return to an original shape after the applied force or pressure is removed.

In some examples, a relatively higher glass transition temperature may be indicative of higher hardness. A material having a relatively lower glass transition temperature or a relatively lower melting temperature may be relatively softer than a material having a relatively higher glass transition temperature or a relatively higher melting temperature. A softer material may be able to endure a higher radial ratio, for example, by having a higher maximum radial ratio than a harder material. In these examples, inner layer 34 may include a material having a glass transition temperature or a melting temperature respectively lower than the glass transition temperature or the melting temperature of outer layer 32 by at least about 10° C. to about 30° C. or greater, such as by at least about 10° C., 15° C., about 20° C., or about 30° C.

While the relative hardness of different materials can be compared in terms of their glass transition temperatures, hardness may also be evaluated in terms of Shore hardness established using a durometer. The shore hardness may include Shore A hardness (for relatively softer materials) or Shore D hardness (for relatively harder materials) as appropriate. In some examples, inner layer 34 has a lower Shore durometer hardness than that of outer layer 32. For example, outer layer 32 may have a hardness of about 74 Shore D, and inner layer 34 may have a hardness of about 40 Shore D, although layers 32, 34 may have other Shore durometer hardnesses in other examples. The Shore durometer hardness may be determined using a durometer and one or both of the Shore A hardness scale or the Shore D hardness scale. In some examples, inner layer 34 is more flexible than outer layer 32. In some examples, inner layer 34 has a lower stiffness than outer layer 32.

The compliance of a balloon layer is the degree or extent to which a balloon expands in response to inflation pressure. A relatively compliant layer (also called a compliant layer herein) is a layer including a material that inflates, deflates or deforms without resulting in mechanical failure of the material. A compliant layer, for example, a layer including a polyether block amide (PEBA) may exhibit stretching in response to an inflationary pressure. In contrast, a non-compliant layer, for example, a layer including a high density polyethylene (HDPE) may exhibit reduced or relatively no stretching compared to a compliant layer. Thus, a non-compliant layer may be a layer that has lower flexibility, lower softness, higher rigidity, or non-compliance to expansion or inflation compared to a compliant layer, for example, resulting in mechanical failure of the material in response to inflation or deformation beyond a predetermined threshold.

Whether a layer is compliant or non-compliant may depend on the composition, hardness, and dimensions, for example, thickness, of the layer. Compliance may be measured, for example, by measuring radial expansion of a layer as a ratio of inflation pressure. In some examples, a compliant layer may exhibit an expansion greater than about 10 millimeters/atmospheres (mm/atm), or greater than about 20 millimeters/atmospheres, or greater than about 50 millimeters/atmospheres. In some examples, a non-compliant layer may exhibit an expansion lower than about 0.02 mm/atm, or lower than about 0.01 mm/atm, or lower than about 0.001 mm/atm. A semi-compliant layer may exhibit an expansion greater than about 0.02 mm/atm and less than about 10 mm/atm. In some examples, inner layer 34 may include a compliant or a semi-compliant layer. In some examples, inner layer 34 may be more compliant than outer layer 32.

The layers of balloon 14 may be formed from any suitable materials that provide the properties described herein. In some examples, one or both of outer layer 32 and inner layer 34, or another layer of balloon 14, may include one or more of a thermoplastic, an elastomer, or an elastomeric thermoplastic. In some examples, one or both of outer layer 32 and inner layer 34, or another layer of balloon 14, may include one or more of acrylonitrile-butadiene styrene (ABS), polyamides, for example, nylons, polyamide 6 (PA 6), or polyamide 66 (PA 66), polycarbonates (PC), polyethylenes (for example, high density polyethylenes (HDPE) or low density polyethylenes (LDPE)), poly(methyl methacrylate) (PMMA), polyoxymethylene (POM), polypropylenes (PP), polystyrenes (PS), polybutylene terephthalate (PBT), styrene acrylonitrile resin (SAN), thermoplastic elastomers (TPE) (for example, polyether block amides (PEBAs)), polyphenylene sulfide (PPS), polyetheretherketones (PEEK), polyurethanes, polyesters, or blends, copolymers, or coextrusions thereof. For example, one or more of outer layer 32 and inner layer 34 may include sublayers, for example, coextruded layers. In some examples, the TPEs (or PEBAs) may include materials sold under the PEBAX® brand name (Arkema, Paris, France) or VESTAMID (Evonik Industries, Essen, Germany). In some examples, the thermoplastic may include materials sold under the GRILAMID® brand name (EMS-Grivory, Domat/Ems, Switzerland), which includes amide thermoplastics.

In examples in which balloon 14 includes coextruded outer layer 32 and inner layer 34, outer layer 32 and inner layer 34 may be fused to each other at respective interfaces as a result of the coextrusion. The fusion may help outer layer 32 and inner layer 34 resist delamination from each other, e.g., during inflation of balloon 14 during a medical procedure.

In some examples, outer layer 32 includes a biaxially oriented thermoplastic. For example, the biaxially oriented thermoplastic may include one or more of a polyamide, a nylon 12, a nylon 6/12, a nylon 610, a nylon 612, or a nylon 1010, a polyester, a polyethelene terephthalate, or a polyurethane. In some examples, one or both of outer layer 32 and inner layer 34 includes a thermoplastic elastomer. For example, the thermoplastic elastomer may include a polyether block amide (PEBA). Any suitable combination of materials may be used for outer layer 32 and inner layer 34. For example, outer layer 32 may include PEBAX 7033 when inner layer 34 includes PEBAX 6333, outer layer 32 may include PEBAX 6333 when inner layer 34 includes PEBAX 5533, or outer layer 32 may include PEBAX 7433 when inner layer 34 includes PEBAX 4033. In some examples, outer layer 32 includes GRILAMID L25 and inner layer 34 includes PEBAX 7033. In such examples, outer layer 32 may have a maximum radial ratio of at least about 5 or 6, and inner layer 34 may have a maximum radial ratio of at least about 6 or 7. In some examples in which outer layer 32 includes GRILAMID L25 and inner layer 34 includes PEBAX 7033, the ratio of the tensile strength (for example, ultimate tensile strength) of the inner layer to that of the outer layer is about 1.75, while achieving a burst pressure (for example, 4255.65 kPA, or 42 atm) similar to the burst pressure of a single layer balloon including GRILAMID 25, and while having a wall thickness (for example, 0.0584 mm, or 0.0023 inches) that is 4.5 times thinner than the wall thickness of the single layer balloon.

Outer layer 32 and inner layer 34 may have any suitable thickness subject to the wall thickness of wall 15 of balloon 14 and to the respective radial ratios of outer layer 32 and inner layer 34. In some examples, inner layer 34 may have a thickness that is between 5% and 50% of the thickness of wall 15 (“T” in FIG. 3). In some examples, the thicknesses for outer layer 32 and inner layer 34 may vary within respective ranges, for example, between 10% and 40% of thickness T of wall 15 for inner layer 34, and between 60% and 90% of thickness T of wall 15 for outer layer 32. The relative thicknesses of outer layer 32 and inner layer 34 may depend on materials and performance requirements. The wall thickness T of wall 15 is the shortest distance between the exterior surface of balloon 14 and the interior surface of balloon 14. For example, the wall thickness T is half the difference between the outer diameter of balloon 14 and the inner diameter of balloon 14. Thus, the wall thickness may be measured across wall 15 along any line extending through a center of balloon 14, or along a line extending along a normal to a surface of balloon 14. In some examples, balloon 14 has a wall thickness of less than 0.0635 mm (0.0025 inches). For example, balloon 14 may have a wall thickness of less than 0.05842 mm (0.0023 inches), or less than 0.0508 mm (0.002 inches), or less than 0.0381 mm (0.0015 inches), or less than 0.0254 mm (0.0010 inches). In some examples, balloon 14 has a burst pressure of at least 1013.25 kPa (10 atmospheres, atm). For example, balloon 14 may have a burst pressure of at least 1519.88 kPa (15 atm), or at least 2026.5 kPa (20 atm), or at least 2533.13 kPa (25 atm), or at least 3039.75 kPa (30 atm), or at least 4053 kPa (40 atm), or at least 4255.65 kPa (42 atm). Balloon 14 may have the aforementioned burst pressures in examples in which balloon has the wall thickness of less than 0.0635 mm (0.0025 inches). For example, balloon 14 may have a thickness of less than or equal to 0.05842 mm (0.0023 inches) and a burst pressure of at least 4053 kPa (40 atm).

In some examples, a compliance of a layer of balloon 14 may be reduced by adding components, for example, reinforcing material or fibers that resist stretching or inflation. For example, one or both of outer layer 32 or inner layer 34 may include one or more reinforcing components, materials, or fibers. In some examples, the reinforcing components may include one or more of glass, metal, alloy, carbon, or polymers.

The multilayer configuration of balloon 14 shown in FIG. 3 may exhibit higher burst or puncture resistance compared to a single layer balloon having the same respective effective wall thickness as respective balloon wall 15 of balloon 14. Thus, example multilayer balloons according to the disclosure may be used in procedures where robustness and puncture-resistance of balloons is desired, while having relatively lower wall thickness.

Balloon 14, as well as other example multilayer balloons according to the disclosure, including an outer layer and an inner layer having a higher maximum radial ratio than that of the outer layer can be formed using any suitable technique. For example, balloon 14 may be formed using any suitable mold assembly, for example, a mold assembly including mold 38 shown in FIG. 4. FIG. 4 is a schematic and conceptual cross-sectional view of mold 38, in which a tube 13 can be expanded to form balloon 14 of FIG. 3. Tube 13 is positioned within mold 38 in the example shown in FIG. 4. A mold assembly (not shown) including mold 38 may be used to form balloon 14 from tube 13, for example, by expanding tube 13 under predetermined conditions (e.g., predetermined conditions of temperature, pressure, and stretching) within mold 38 of the mold assembly. Thus, tube 13 is a structure from which balloon 14 may be formed.

Mold 38 is substantially rigid, and an inner surface 39 of mold 38 constrains tube 13 as it is expanded under the predetermined conditions of temperature, pressure, and stretching. Mold 38 may be formed of any suitable material including one or more of a metal, an alloy, a ceramic, a glass, or a plastic, or another rigid material capable of constraining the expansion of tube 13 beyond mold inner diameter IDₘ. Mold 38 defines the shape of balloon 14 formed by expanding tube 13. In some examples, mold 38 may be substantially cylindrical, and tube 13 expanded within cylindrical mold 38 may form a substantially cylindrical balloon 14. However, mold 38 may have any suitable shape complementary to a target shape of balloon 14 formed from tube 13. For example, one or more portions of mold 38 may define conical surfaces, or dome-shaped surfaces, or other curved or flat surfaces to respectively define conical, dome-shaped, or other curved or flat surfaces of balloon 14. In some examples, the ends of mold 38 may define conical portions, and the portion of mold 38 extending between the ends may define a cylindrical portion. Mold 38 may also include a substrate, for example a mandrel, on which tube 13 may extruded or positioned and held during expansion within mold 38.

Prior to being expanded within mold 38, tube 13 may include an initial configuration of outer layer 32 and inner layer 34. In an initial configuration (prior to expansion within mold 38), tube 13 includes coextruded outer layer 32 and inner layer 34 in an unblown or non-stretched configuration. In the initial configuration shown in FIG. 4, outer layer 32 has a first inner diameter ID₁, and inner layer 34 has a second inner diameter ID₂. Mold 38 defines an inner surface 39 having a mold inner diameter IDₘ.

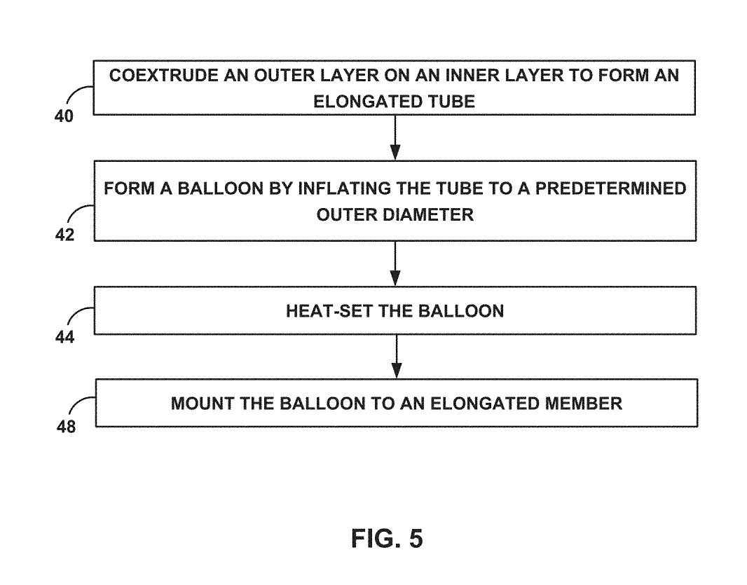

FIG. 5 is a flow diagram illustrating an example technique for manufacturing balloon 14. The example technique of FIG. 5 is described with reference to mold 38 of FIG. 4. However, the example technique of FIG. 5 may be implemented using any suitable mold. In some examples, the example technique of FIG. 5 may include coextruding outer layer 32 on inner layer 34 (40). For example, respective heated, flowable, or molten compositions for outer layer 32 and inner layer 34 may be coextruded from an extrusion die onto a substrate, for example, a mandrel. Coextrusion of multiple layers may result in a uniform balloon structure. For example, coextruding layers of balloon 14 simultaneously may provide respective layers with a substantially uniform layer thickness and thus uniform thickness of wall 15 along a length of balloon 14. In some examples, providing a uniform thickness to wall 15 may provide balloon 14 with a substantially uniform diameter along a longitudinal axis in an inflated configuration.

The example technique of FIG. 5 may include coextruding tube 13 including outer layer 32 and inner layer 34. In other examples, outer layer 32 and inner layer 34 may be coextruded as a multilayer sheet, for example, by coextruding onto a flat substrate, and tube 13 may be subsequently formed from the sheet.

In other examples of the technique shown in FIG. 5, outer layer 32 and inner layer 34 may be sequentially extruded. In some examples, a pair of layers may be coextruded, followed by extrusion of another layer. For example, outer layer 32 and inner layer 34 may be coextruded, followed by extrusion of another layer on the coextruded structure. In some examples, inner layer 34 may be extruded, followed by extrusion of outer layer 32 on inner layer 34. In some examples, the order of layers during extrusion or coextrusion may be different from the order of layers in balloon 14. For example, in examples in which balloon 14 includes three or more layers, pairs or groups of layers may be coextruded, and reordered, stacked or otherwise combined in a mold followed by pressurizing in the mold to eventually form balloon 14.

Balloon 14 may be formed from multilayer tube 13 or a multilayer sheet including outer layer 32 and inner layer 34 (42). For example, tube 13 may be placed or secured in mold 38 configured to provide the shape of balloon 14, as shown in FIG. 4. Tube 13 may be blown by applying pressure in addition to axial or radial stretching, so that tube 13 expands to occupy the periphery of the mold and contacts inner surface 39. Tube 13 may be heated to a temperature greater than one or both of the respective glass transition temperatures of outer layer 32, or inner layer 34, or another suitable temperature, to soften tube 13. Blown tube 13 may expand to an outer diameter substantially the same as mold inner diameter IDₘ defined by mold 38 before tube 13 is cooled or otherwise cured or solidified. The ratio IDₘ/ID₁ may define a radial ratio of outer layer 32, and the ratio IDₘ/ID₂ may define a radial ratio of inner layer 34. As described in the disclosure, the ratio of inner diameter ID₂ of tube 13 to the mold inner diameter IDₘ may be linked to the respective internal orientations within outer layer 32 and inner layer 34 in the final balloon 14. One or more of IDₘ, ID₁, or ID₂ may be selected such that the respective radial ratios of outer layer 32 and inner layer 34 are less than the respective maximum radial ratios of outer layer 32 and inner layer 34, for example, to prevent premature rupture, tearing, or burst of balloon 14 during manufacture of balloon 14.

In some examples, the respective compositions for outer layer 32 and inner layer 34 may be directly coextruded into mold 38, so that balloon 14 is shaped during coextrusion. In some examples, one or both of inner layer 34 or outer layer 32 may be extruded onto a reinforcing substrate, for example, a reinforcing fabric, an or an arrangement of reinforcing components or fibers. In some examples, reinforcing components may be introducing during the coextrusion.

The coextruding (40) may include stretching balloon 14, for example, one or both of axial or radial stretching. For example, a region or side of balloon 14 may be intermittently heated or stretched during or after the coextruding. In some examples, the stretching may include double stretching, or stretching balloon 14 from two sides. In some examples, the stretching may include a primary stretching at a first pressure followed by a secondary stretching at a second pressure. The stretching may promote a uniform wall thickness and promote uniform inflation of balloon 14.

In some examples, the example technique of FIG. 5 includes heat-setting balloon 14 (44). In some examples, heat-setting may include annealing, for example, heating and maintaining balloon 14 at a predetermined temperature for a predetermined period of time. The predetermined temperature may be near or above a melt transition of one or both of outer layer 32 or inner layer 34, or near or above a glass transition temperature of one or more polymers in one or both of outer layer 32 or inner layer 34. Heat-setting may help remove creases, wrinkles, or marks from surfaces of balloon 14, and may further provide a uniform thickness to a wall of balloon 14, for example, wall 15 of balloon 14. For example, heat-setting may also be used to control the wall thickness of wall 15 of balloon 14.

Heat-setting may be performed using any suitable technique. For example, balloon 14 may be heated in mold 38, such that the heat-setting may provide a permanent set or shape for balloon 14. Heat-setting may be used to control compliance of one or both of outer layer 32 or inner layer 34, or overall compliance and burst resistance of balloon 14. In some examples, the configuration of balloon 14 as molded and heat-set may correspond to an uninflated configuration of balloon 14. In some examples, the configuration of balloon 14 as molded and heat-set may correspond to an inflated configuration of balloon 14.

In some examples, the example technique of FIG. 5 may further include securing second medical device 26 to balloon 14. For example, in examples in which second medical device 26 includes a stent, securing second medical device 26 to the balloon may include crimping the stent to balloon 14.

In some examples, the example technique of FIG. 5 may further include mounting balloon 14 to elongated member 12 (46). For example, elongated member 12 may include a catheter body, and balloon 14 may be mounted to the catheter body.

While the example technique of FIG. 5 may be used to prepare balloon 14, other techniques for forming balloon 14 may be used in other examples.

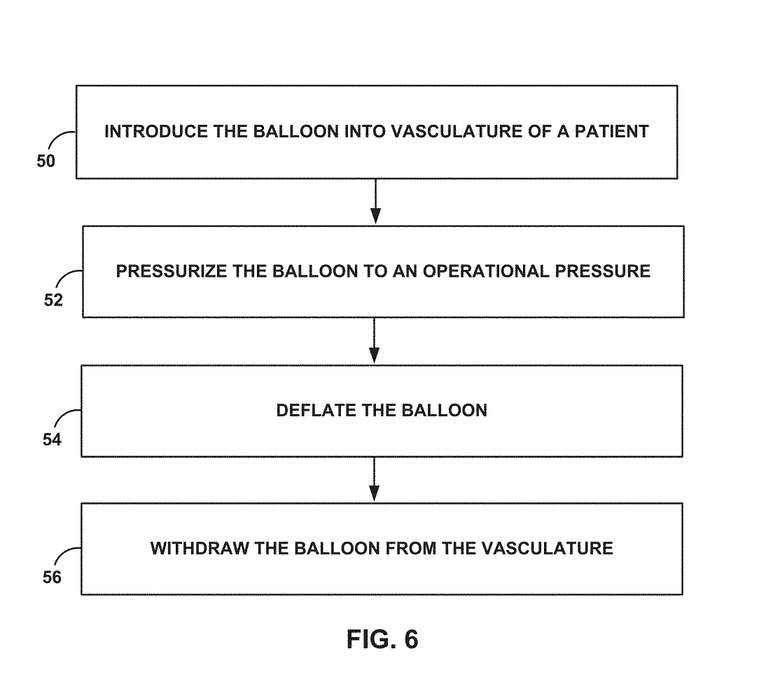

FIG. 6 is a flow diagram illustrating an example technique for using balloon 14 in a procedure. In some examples, the example technique of FIG. 6 includes introducing balloon 14 into vasculature of a patient (50). For example, distal tip 16 of elongated member 12 may be introduced at an incision or body opening and into the vasculature, followed by the shaft of elongated member 12 carrying balloon 14. In some examples, introducing balloon 14 into the vasculature (50) may include advancing balloon 14 carried on elongated member 12 over a guidewire or other guide member through the vasculature to a target site within the vasculature. In examples in which balloon 14 includes radiopaque marker 17, a clinician may use radiopaque marker 17 to visualize the position of balloon 14 relative to the target site within the vasculature, for example, by radioimaging.

After balloon 14 arrives at the target site, balloon 14 may be inflated, such as by pressurizing the balloon to an operational pressure (52). The operational pressure may be a pressure sufficient to inflate balloon 14 to an operational dimension, for example, an operational diameter. For example, the operational diameter may be an average diameter of balloon 14 in an inflated configuration that is sufficient to expand, clear, or scaffold a region of the vasculature adjacent the target site. In some examples, the operational diameter may be a diameter sufficient to deploy second medical device 26 at the target size, for example, by causing second medical device 26 to expand, move, or decouple from balloon 14 or elongated member 12, and occupy the target site.

In some examples, the example technique of FIG. 6 includes, after the pressurizing (52), deflating balloon 14 (54). A clinician may, for example, withdraw inflating fluid from inflating lumen port 22 to cause balloon 14 to depressurize and contract, shrink, collapse, fold, or otherwise attain a compact configuration allowing safe withdrawal of balloon 14a from the vasculature. After deflating balloon 14 (54), the clinician may withdraw balloon 14 from the vasculature (56). For example, elongated member 12 carrying balloon 14 may be withdrawn from the vasculature.

While the example technique of FIG. 6 is described with respect to the vasculature, the example technique of FIG. 6 may be used to advance and deploy balloon 14 at a target site within any body lumen accessible through a body opening or incision. Thus, an example technique for using balloon 14 has been described with reference to FIG. 6.

Thus, the disclosure describes multilayer balloons with higher burst resistance at lower wall thicknesses compared to single layer balloons, and techniques for forming and using multilayer balloons. Balloons having a higher burst pressure may be more robust than balloons having relatively lower burst pressure, for example, by resisting rupture or tearing during transport to or deployment at the target site. In some examples, achieving the high burst pressure with relatively low wall thickness may provide example balloons according to the disclosure with increased deliverability to a target site within a patient, for example, a target site within vasculature of the patient. For example, providing a low wall thickness may allow the balloon to be relatively low profile and flexible, enabling the balloon to conform to bends and exhibit relatively low resistance to travel through tortuous paths of the vasculature. Providing a smaller wall thickness may also reduce the profile of the balloon, allowing the balloon to be deployed through relatively narrow lumens of the vasculature or other anatomical features.

In some examples, providing an inner layer with a higher maximum radial ratio than an outer layer in a balloon may provide the inner layer with increased flexibility or compliance to stretching, which may result in increased deliverability of the balloon. However, if the balloon only includes a single layer having a relatively high maximum radial ratio, then the balloon may inflate beyond a target inflation diameter. The target inflation diameter may be selected to, for example, help avoid undesirable distention of a blood vessel. In contrast, in a multi-layer balloon, providing an outer layer having a lower maximum radial ratio may help constrain inflation of the balloon to within predetermined target inflation diameters. For example, providing the outer layer of a multilayer balloon with a lower maximum radial ratio may make the outer layer relatively harder and less compliant than the inner layer, and the outer layer may resist and prevent inflation of the balloon beyond the target inflation diameter. Thus, in some examples, even if a clinician attempts to exert relatively high inflation pressure on an example balloon according to the disclosure, the balloon may not exhibit significant change in the diameter beyond the target inflation diameter.

The example multilayer balloons described herein may be a part of any suitable medical device, and can be used for any suitable medical procedure, such as, but not limited to, angioplasty (e.g., plain balloon angioplasty (POBA)), stent delivery, heart valve delivery, other dilatation uses, vascular occlusion, and the like.

### EXAMPLES

### Comparative Example 1

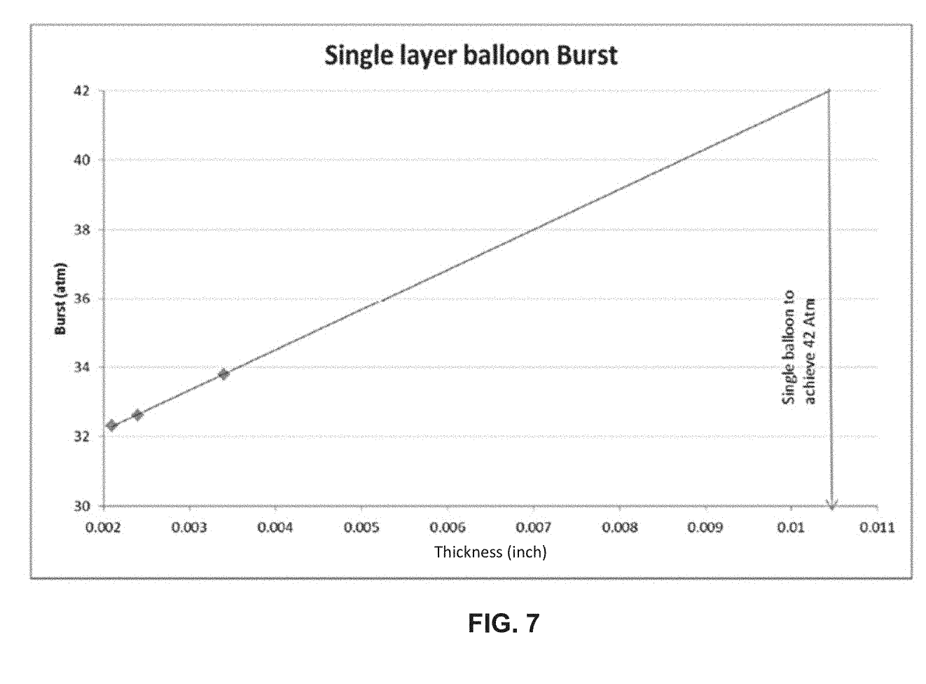

The relation between pressure at burst and wall thickness for single layer balloons was evaluated. The balloons included nylon 12 material (Grilamid L25, available from EMS-Grivory, Domat/Ems, Switzerland). Balloons having different wall thicknesses were inflated until they burst, and the pressure at burst was recorded for each balloon. The results are shown in FIG. 7. FIG. 7 is a graph illustrating a relationship between burst pressure and wall thickness for sample single layer balloons. As seen in FIG. 7, an increase in the burst pressure requires an increase in the wall thickness. Extrapolation indicates that a balloon wall thickness of 0.2667 mm (0.0105 inches) would be required to achieve a burst pressure of 4255.65 kPa (42 atmospheres).

### Example 1

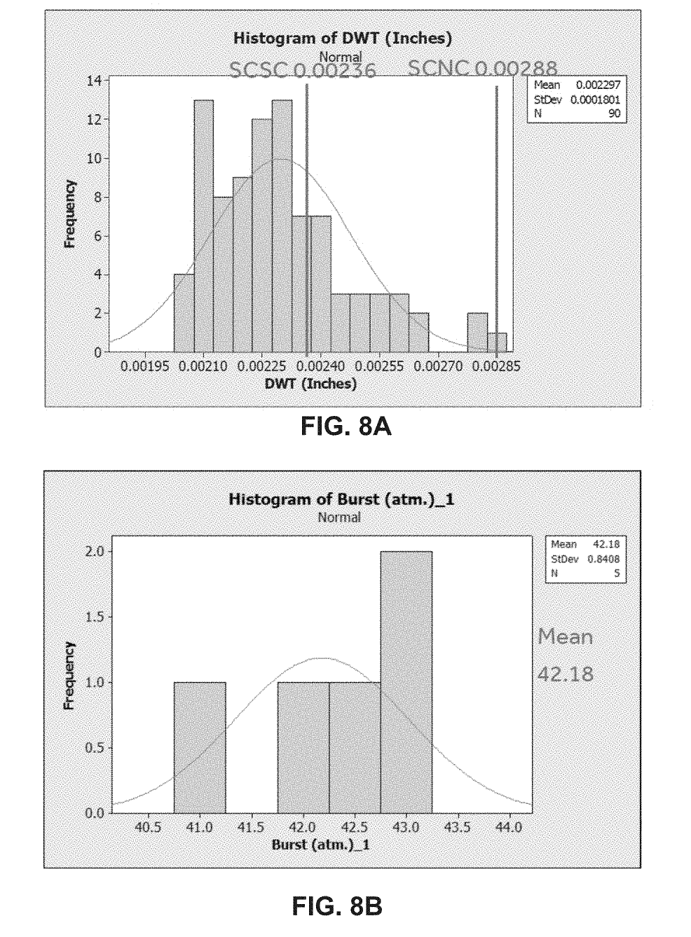

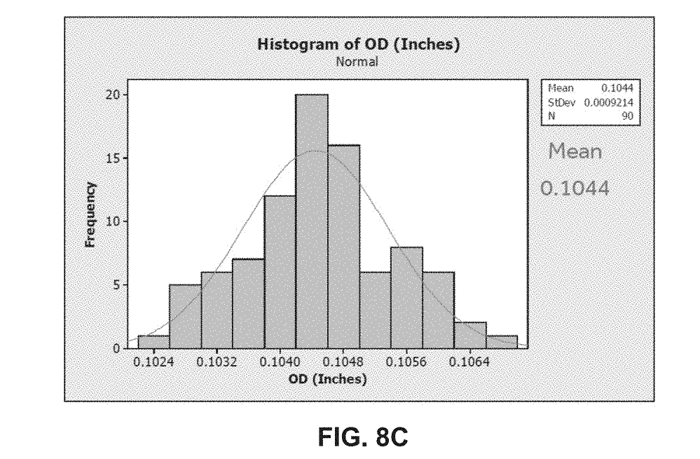

The relation between pressure at burst and wall thickness for double layer balloons was evaluated. The balloons included an inner layer of PEBAX 7033 (available from Arkema, King of Prussia, Pa.) and an outer layer of Grilamid L25. A number of example balloons were prepared by expanding a tube including the inner layer and the outer layer in a mold. FIG. 8A is a histogram illustrating wall thicknesses of a sample population of the double layer balloons. FIG. 8B is a histogram illustrating burst pressures of a sample population of the double layer balloons. FIG. 8C is a histogram illustrating outer diameters of a sample population of double layer balloons.

As respectively seen in FIGS. 8A, 8B, and 8C, the mean double wall thickness (DWT) of the population was 0.0584 mm (0.0023 inches), the mean burst pressure was 42 atmospheres, and the mean outer diameter (OD) was 2.652 mm (0.1044 inches). Thus, a double wall thickness of 0.0584 mm (0.0023) inches was sufficient to achieve a burst pressure of 4255.65 kPa (42 atmospheres), which was significantly thinner than the wall thickness of 0.2667 mm (0.0105 inches) of the single layer balloon of COMPARATIVE EXAMPLE 1 required for a similar burst pressure.

### Example 2

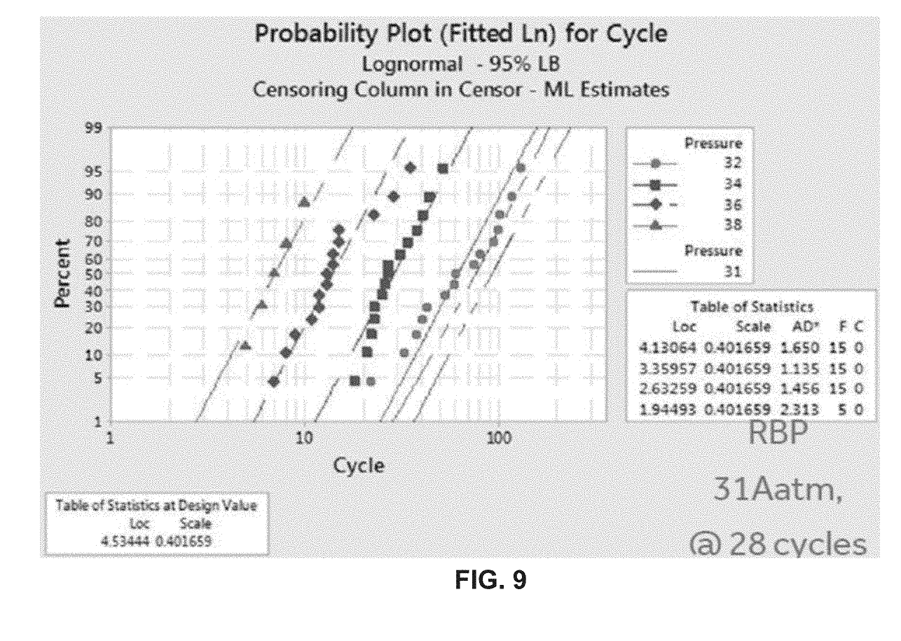

The effect of subjecting balloons to different pressure cycles was evaluated. FIG. 9 is a graph illustrating probability of burst at different pressures for balloons subjected to cycling. Accelerated life testing (ALT) may be used to estimate the fatigue life of a balloon upon repeated inflation/deflation at a set pressure. Burst may be an indicator of the fatigue capability. ALT may be used to determine a statistical confidence level in the balloon meeting a certain number of cycles at certain pressure levels. As seen in FIG. 9, the multilayer balloon of EXAMPLE 1 is capable of sustaining 28 inflation/deflation cycles at 3141 kPa (31 Atmosphere, atm).

Various examples have been described. These and other examples are within the scope of the following claims.

What is claimed is:

1. A medical device comprising:
a balloon inflatable to an inflated configuration, the balloon comprising:
an inner layer; and
an outer layer coextruded on the inner layer, wherein the outer layer has a maximum radial ratio that is lower than that of the inner layer.

2. The medical device of claim 1, wherein the inner layer has a first maximum radial ratio of up to 8.5, and wherein the outer layer has a second maximum radial ratio of up to 6.5.

3. The medical device of claim 1, wherein the inner layer comprises a first material having a first glass transition temperature, and wherein the outer layer comprises a second material having a second glass transition temperature higher than the first glass transition temperature.

4. The medical device of claim 1, wherein the inner layer has a lower Shore durometer hardness than that of the outer layer.

5. The medical device of claim 1, wherein the inner layer is more flexible than the outer layer.

6. The medical device of claim 1, wherein the balloon has a wall thickness of less than 0.0635 mm (0.0025 inches).

7. The medical device of claim 6, wherein the balloon has a burst pressure of at least 1013 kPa (10 atmospheres).

8. The medical device of claim 7, wherein the balloon has a burst pressure of at least 4053 kPa (40 atmospheres).

9. The medical device of claim 1, wherein the inner layer has a lower stiffness than the outer layer.

10. The medical device of claim 1, wherein the outer layer comprises a biaxially oriented thermoplastic.

11. The medical device of claim 10, wherein the biaxially oriented thermoplastic comprises one or more of a polyamide, a nylon 12, a nylon 6/12, a nylon 610, a nylon 612, or a nylon 1010, a polyester, a polyethelene terephthalate, or a polyurethane.

12. The medical device of claim 1, wherein at least one of the inner layer or the outer layer comprises a thermoplastic elastomer.

13. The medical device of claim 12, wherein the thermoplastic elastomer comprises a polyether block amide (PEBA).

14. A system comprising the medical device of claim 1 and a second medical device secured to the balloon.

15. The system of claim 14, wherein the second medical device comprises a stent crimped to the balloon.

16. The medical device of claim 1, further comprising an elongated member, wherein the balloon is mounted to the elongated member, wherein the elongated member comprises a catheter body.

17. A medical device comprising:
a balloon inflatable to an inflated configuration, the balloon comprising:
an inner layer, the inner layer comprising a first material having a first glass transition temperature; and
an outer layer coextruded on the inner layer, the outer layer comprising a second material having a second glass transition temperature higher than the first glass transition temperature, the balloon having a wall thickness of less than 0.0635 mm (0.0025 inches), and the balloon having a burst pressure of at least 4053 kPa (40 atmospheres).

18. The medical device of claim 17, wherein the inner layer has a first maximum radial ratio of up to 8.5, and wherein the outer layer has a second maximum radial ratio of up to 6.5.

19. A method comprising:
coextruding an outer layer on an inner layer to form an elongated tube, wherein the outer layer has a maximum radial ratio that is lower than that of the inner layer; and
forming a balloon by at least expanding the elongated tube within a mold defining a predetermined outer diameter of the balloon.

20. The method of claim 19, wherein forming the balloon further comprises molding the inner layer and the outer layer over a scaffold.

21. The method of claim 19, wherein forming the balloon further comprises heat-setting the balloon.

22. The method of claim 19, further comprising securing a medical device to the balloon.

23. The method of claim 22, wherein securing the medical device to the balloon comprises crimping a stent to the balloon.

24. A method comprising:
introducing a balloon into vasculature of a patient, wherein the balloon comprises an outer layer coextruded on an inner layer, the outer layer having a maximum radial ratio that is lower than that of the inner layer; and
after introducing the balloon into the vasculature, pressurizing the balloon to an operational pressure.

25. The method of claim 24, further comprising, after pressurizing the balloon, deflating the balloon, and withdrawing the balloon from the vasculature.
